SEQUENCE LISTING (40 sequences)

SEQ ID NO: 1 - DNA, 22 bp, Artificial Sequence
  Synthetic oligonucleotide
cggccgctttgtctacacagct

SEQ ID NO: 2 - DNA, 28 bp, Artificial Sequence
  Synthetic oligonucleotide
agtttctcatggcactcaaaataggcct

SEQ ID NO: 3 - DNA, 28 bp, Artificial Sequence
  Synthetic oligonucleotide
gccgggtcatgttaattgggaaaaagaa

SEQ ID NO: 4 - DNA, 27 bp, Artificial Sequence
  Synthetic oligonucleotide
tccagtccttcgtcaaagactgcctta

SEQ ID NO: 5 - DNA, 22 bp, Artificial Sequence
  Synthetic oligonucleotide
caacgggtcgcagcccttgctg

SEQ ID NO: 6 - DNA, 23 bp, Artificial Sequence
  Synthetic oligonucleotide
tgagacaggagtctgagcagggc

SEQ ID NO: 7 - DNA, 25 bp, Artificial Sequence
  Synthetic oligonucleotide
ctcctcctgcactgctatgcagatt

SEQ ID NO: 8 - DNA, 28 bp, Artificial Sequence
  Synthetic oligonucleotide
ggcagtcaagctatcctctttcctcttt

SEQ ID NO: 9 - DNA, 31 bp, Artificial Sequence
  Synthetic oligonucleotide
ctctggaaaacaatcaactaagccctttgac

SEQ ID NO: 10 - DNA, 26 bp, Artificial Sequence
  Synthetic oligonucleotide
actctacgcgctatataagcaggcca

SEQ ID NO: 11 - DNA, 21 bp, Artificial Sequence
  Synthetic oligonucleotide
agcagttttgcaagggatgcc

SEQ ID NO: 12 - DNA, 24 bp, Artificial Sequence
  Synthetic oligonucleotide
gtagcctctggaatccgtagtcaa

SEQ ID NO: 13 - DNA, 25 bp, Artificial Sequence
  Synthetic oligonucleotide
gcatgttgtccatggaacaagtttc

SEQ ID NO: 14 - DNA, 21 bp, Artificial Sequence
  Synthetic oligonucleotide
gctctttcgtcactctggcct

SEQ ID NO: 15 - DNA, 21 bp, Artificial Sequence
  Synthetic oligonucleotide
tacaagatggttgccgggtca

SEQ ID NO: 16 - DNA, 25 bp, Artificial Sequence
  Synthetic oligonucleotide
cagacttctccaaatgtttcaactt

SEQ ID NO: 17 - DNA, 20 bp, Artificial Sequence
  Synthetic oligonucleotide
cacatgggcttctcggggga

SEQ ID NO: 18 - DNA, 22 bp, Artificial Sequence
  Synthetic oligonucleotide
cgtctcgtagcgcttgatgaag

SEQ ID NO: 19 - DNA, 20 bp, Artificial Sequence
  Synthetic oligonucleotide
gccagagctcagagtggcaa

SEQ ID NO: 20 - DNA, 20 bp, Artificial Sequence
  Synthetic oligonucleotide
ctgggtgagggcgtctctgg

SEQ ID NO: 21 - DNA, 22 bp, Artificial Sequence
  Synthetic oligonucleotide
cggatcatctctgcacctgaga

SEQ ID NO: 22 - DNA, 21 bp, Artificial Sequence
  Synthetic oligonucleotide
ctttgagctcctgcagggaag

SEQ ID NO: 23 - DNA, 20 bp, Artificial Sequence
  Synthetic oligonucleotide
ctcatgctggcggccctcct

SEQ ID NO: 24 - DNA, 24 bp, Artificial Sequence
  Synthetic oligonucleotide
atggtcttttcaaccatgtcctcc

SEQ ID NO: 25 - DNA, 23 bp, Artificial Sequence
  Synthetic oligonucleotide
ctgctttctctgagaggcagtca

SEQ ID NO: 26 - DNA, 22 bp, Artificial Sequence
  Synthetic oligonucleotide
gcccgccagaaaaacatgaaga

SEQ ID NO: 27 - DNA, 25 bp, Artificial Sequence
  Synthetic oligonucleotide
ggatccctctcaattatctctggaa

SEQ ID NO: 28 - DNA, 23 bp, Artificial Sequence
  Synthetic oligonucleotide
cctgcagtaacagaactcactgc

SEQ ID NO: 29 - DNA, 25 bp, Artificial Sequence
  Synthetic oligonucleotide
caggaaacgaagtgaagctaaaact

SEQ ID NO: 30 - DNA, 23 bp, Artificial Sequence
  Synthetic oligonucleotide
agtcaaatacacctggtttgggc

SEQ ID NO: 31 - DNA, 24 bp, Artificial Sequence
  Synthetic oligonucleotide
tccgtagtcaatggtgggctcgtt

SEQ ID NO: 32 - DNA, 27 bp, Artificial Sequence
  Synthetic oligonucleotide
ggcccaacaagatcatccagggaattc

SEQ ID NO: 33 - DNA, 31 bp, Artificial Sequence
  Synthetic oligonucleotide
caacttctttagttcaacctcttcctgtgga

SEQ ID NO: 34 - DNA, 31 bp, Artificial Sequence
  Synthetic oligonucleotide
agcttattatactttggtctcttcctgtgat

SEQ ID NO: 35 - DNA, 22 bp, Artificial Sequence
  Synthetic oligonucleotide
cccacgctgtctgccaccgtca

SEQ ID NO: 36 - DNA, 22 bp, Artificial Sequence
  Synthetic oligonucleotide
ggaagcctccatgtgtctgcgg

SEQ ID NO: 37 - DNA, 29 bp, Artificial Sequence
  Synthetic oligonucleotide
tcttttcaaccatgtcctccaggagtttg

SEQ ID NO: 38 - DNA, 31 bp, Artificial Sequence
  Synthetic oligonucleotide
agaaaaacatgaagaaaacccaggaaattag

SEQ ID NO: 39 - DNA, 25 bp, Artificial Sequence
  Synthetic oligonucleotide
tcactgcatttgaggtcaagctgct

SEQ ID NO: 40 - DNA, 26 bp, Artificial Sequence
  Synthetic oligonucleotide
tggtttgggcatgttacaaggtcctt

(19) United States
(12) Patent Application Publication
(10) Pub. No.: US 2010/0297622 A1
(43) Pub. Date: Nov. 25, 2010

(54) Method for high-throughput gene expression profile analysis

(76) Inventors: Honghua Li, Monmouth Junction, NJ (US); Guohong Hu, Piscataway, NJ (US); Qifeng Yang, Highland Park, NJ (US)

Correspondence Address: Jane Massey Licata;Licata & Tyrrell P.C., 66 E. Main Street, Marlton, NJ, 08053, US

(21) Appl. No.: 12/469,105

(22) Filed: May 20, 2009

(51) Int. Cl. C12Q 1/68

(52) U.S. Cl. 435/6

(57) ABSTRACT

The present invention embraces a method for high-throughput gene profiling with high specificity and sensitivity. With this system, >1000 mRNA species can be co-amplified using gene-specific primers from a single cell. The primers are designed to amplify sequences of desirable length, which are in different exons. The exons can be either adjacent and separated by a large intron or include more than two exons. The amplified sequences are then analyzed by microarray with probes hybridizing to neighboring exons.

This invention was made in the course of research sponsored by the National Human Genome Research Institute, grant number RO1 HG02094; National Cancer Institute, grant number R33 CA 96309; and the National Institutes of Health, grant number RO1 CA77363. The U.S. government has certain rights in this invention.

### BACKGROUND OF THE INVENTION

Biological processes are underlain by interactions between various genes, their products, and defined pathways in the molecular networks. Global gene expression profiling of cells and tissues under physiological or in vitro conditions facilitates the understanding of the correlation between gene function and phenotypic effects. The advent of the microarray-based high-throughput RNA detection system (Schena, et al. (1995) Science 270:467-470; Lockhart, et al. (1996) Nat. Biotechnol. 14(13):1675-1680) has made it possible to profile gene expression patterns for the entire transcriptome. However, to specifically detect individual transcripts, discrimination between closely related sequences, including the genomic sequence, pseudogenes, and unprocessed RNA, is essential. Although contamination with genomic DNA may not be a concern for applications using purified mRNA, gene sequences must be taken into consideration for applications using cell lysate directly without RNA extraction. This becomes especially important when the transcripts under study are present at low abundance. As indicated, pseudogenes and their possible transcripts are also of concern. The number of pseudogenes in the human genome has been estimated to be 20,000 to 33,000 (Goncalves, et al. (2000) Genome Res. 10(5):672-678; Harrison, et al. (2002) Genome Res. 12(2):272-280), and usually share a high degree of sequence identity with the closely related genes.

Among the microarray-based platforms, GENECHIP (AFFYMETRIX, Santa Clara, Calif.) is a commonly used system that has contributed to the understanding of complex gene expression networks. However, because this technology is limited by its high degree of nonspecificity and insensitivity, its application has been limited in molecular network integration. Analysis has indicated that 20,696 (10.5%) probes on the GENECHIP U95A/Av2 Array are nonspecific and 18,363 (9.3%) probes missed the target transcript sequences (Zhang, et al. (2005) Genomics 85(3):297-308). The numbers of nonspecific and mis-targeted probes on the U133A array are comparable at 29,405 (12.1%) and 19,717 (8.0%), respectively (Zhang, et al. (2005) supra). These problematic probes certainly and substantially compromise data accuracy, decrease the value of microarray data, and are not acceptable in the study of molecular network integration.

High-throughput gene expression profiling with superior sensitivity is becoming more and more attractive. For example, in breast cancer research, analysis of specimens from microdissection can provide important information about genes involved in different cancer development stages. In most applications, gene expression profiling with microarrays, including GENECHIP, requires amplification of sample RNA. Conventionally, 1 to 3 μg of RNA is required for each assay. However, specimens from fine needle biopsy typically only contain a limited number of cells. The ability to analyze a large number of genes in single cells would facilitate our understanding of the origin and clonality of cancer development and provide the molecular details involved in different stages of the cell cycle.

Current methodologies for gene expression profiling in small RNA samples, especially those from single cells, are very limited. Many of these protocols require multiple enzymatic reactions that may seriously reduce the sensitivity and compromise the specificity. RNA preparation in most of applications also involves a number of steps, which is rather lengthy, tedious, and requires highly skilled personnel.

A number of strategies have been used to enhance the multiplex amplification capacity. For example, optimization of polymerase chain reaction (PCR) conditions has been used to enhance multiplex amplification capacity. While optimizing PCR conditions may allow more sequences to be amplified simultaneously, the major factor limiting amplification of multiple amplicons is primer-primer interaction and optimized PCR conditions cannot reduce the amount of interaction between primers. For example, when n PCR primer pairs are combined in one reaction, the possible interacting pairs between these primers would be 2n²+n, many of which may result in a nonspecific product.

Another strategy for increasing multiplexing capacity involves the use of universal sequences at the 5′-ends of all specific primers. Attaching these sequences during the early stage of amplification enables multiplex amplification of 26 to >1000 sequences in a single reaction. However, since attaching universal sequences involves multiple enzymatic reactions and does not eliminate primer-primer interactions, amplification capacity and sensitivity are still limited with this approach.

Thus there remains a need for improved gene expression profiling.

### SUMMARY OF THE INVENTION

The present invention embraces a method for high-throughput gene expression profile analysis. More specifically, the invention relates to the design of primers and probes for large-scale gene expression profiling, which provide high specificity and sensitivity. The present method involves the steps of:

(I) synthesizing a plurality of primers and probes for a set of genes of interest by:
(a) determining the intron-exon structure of each gene of interest;
(b) selecting for each gene of interest a probe, wherein said probe:
   (i) is less than 50 bases in length, and
   (ii) anneals to two adjacent exons flanking an intron of at least 200 bases in length;
(c) subsequently selecting for each gene of interest a pair of primers, wherein said primers:
   (i) are less than 50 bases in length,
   (ii) anneal to two exons of the gene of interest,
   (iii) produce an amplicon of less than 200 base pairs in length, and
   (iv) when compared to sequences in one or more databases of genomic or mRNA sequences, fail to amplify a non-specific sequence in silicon and
(d) subsequently selecting a nested primer, wherein said nested primer:
   (i) anneals to the amplicon of step (c)(ii), and
   (ii) produces, in an amplification reaction, a single-stranded nucleic acid molecule hybridizable with the probe of step (b); and

(II) subjecting a sample to an amplification reaction with the plurality of primers of step (I)(c), thereby producing double-stranded amplicons;

(III) amplifying single-stranded nucleic acid molecules from the double-stranded amplicons with the plurality of nested primers of step (I)(d); and

(IV) quantifying the single-stranded nucleic molecules via hybridization with the plurality of probes of step (I)(b) thereby determining the expression profile of the set of genes of interest in the sample.

According to certain embodiments, the probe of the instant method has a melting temperature of between 50° C. and 60° C., has a GC content of between 35% and 70%, complements less than nine contiguous bases or 11 contiguous bases with a gap of any primer or probe of the plurality of primers and probes, and complements at least 14 contiguous bases or 18 contiguous bases with a gap of the gene of interest. In particular embodiments, the probe is labeled.

According to other embodiments, the primers of said pair of primers have a melting temperature of between 46° C. and 56° C., have a GC content of between 35% and 70%, complement less than nine contiguous bases or 12 contiguous bases with a gap of any other primer of the plurality of primers, and complement at least 14 contiguous bases or 18 contiguous bases with a gap of the gene of interest.

Using the method of the invention, primers are specially designed to amplify processed RNA sequences very specifically. Probes used for microarray detection are designed only to hybridize to sequences amplified from processed RNA. A large number of RNA species directly released from very few cells or even single cells can be amplified to a detectable amount without RNA isolation. Amplified products can then be detected by the single-base extension assay on an oligonucleotide microarray.

### BRIEF DESCRIPTION OF THE DRAWINGS

FIG. 1A is a schematic illustration of the high-throughput gene expression profiling procedure.

FIG. 1B is a schematic illustration showing the selectivity of the primers and probes of the invention for processed RNA over genomic DNA, pseudogenes, and unprocessed RNA.

### DETAILED DESCRIPTION OF THE INVENTION

The present invention embraces a method for high-throughput gene expression profile analysis which involves designing primers and probes exhibiting a high degree of specificity and sensitivity. Compared with conventional gene expression profiling methods, the method of the invention is simple, safe, and very flexible and provides high specificity and high sensitivity.

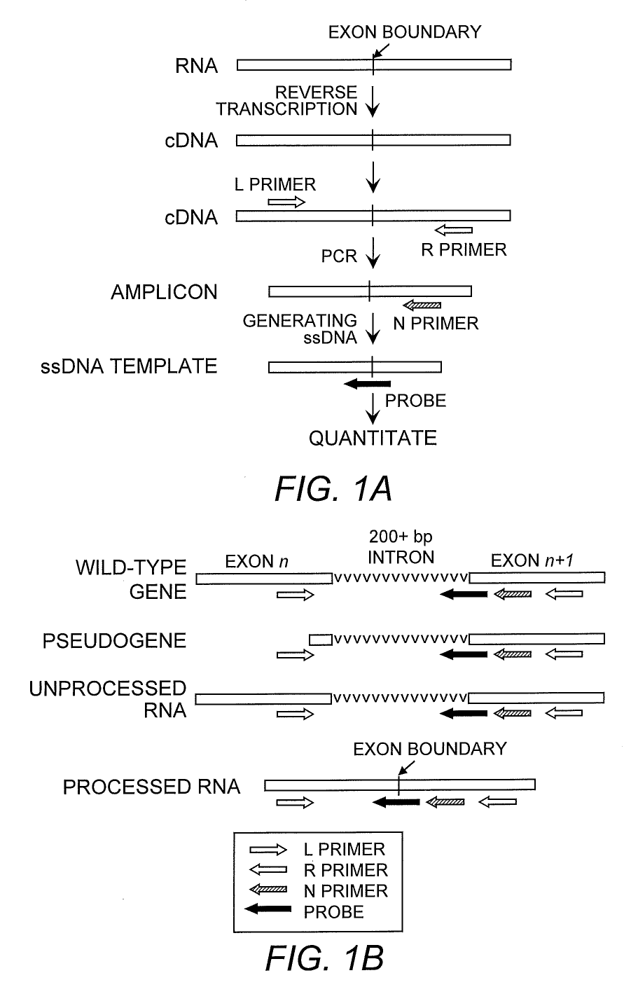

As illustrated in FIG. 1A, the present invention involves the steps of synthesizing a plurality of primers and mRNA-specific probes for a set of genes of interest; subjecting a sample to an amplification reaction with the plurality of primers, thereby producing double-stranded amplicons; amplifying single-stranded nucleic acid molecules from the double-stranded amplicons with a plurality of nested primers; and quantifying the single-stranded nucleic molecules via hybridization with the plurality of probes thereby determining the expression profile of the set of genes of interest in the sample.

The method of the present invention is not limited by the nature of the gene of interest. In this regard, the gene of interest can be any gene, the expression product of which is sought to be detected in a sample. In this regard, a set of genes of interest can include genes encoding proteins or RNA involved in a particular pathological condition, responses to therapeutics, signal transduction, cell proliferation, responses to inflammatory stimuli, and the like. A “set of genes of interest” is intended as two or more genes. A set can be 10 genes, 50 genes, 100 genes or as many as 10000 genes, the expression product of which is sought to be detected in a sample. In accordance with particular embodiments of the present invention, an expression product is an RNA, e.g., a messenger RNA (mRNA), ribosomal RNA (rRNA), transfer RNA (tRNA), small nuclear RNA (snRNA), or microRNA (miRNA).

In so far as 10, 50, 100 or 10000 or more genes or interest are analyzed according to the method of the invention, a “plurality of primers and probes” is commensurate with the number of genes of interest.

The specificity and sensitivity of the instant method is derived from the selection of the primers and probes used in the gene expression profiling. As shown in FIG. 1B, the primers and probes are designed so that they are specific for processed RNA, or amplification products thereof, and not unprocessed RNA, pseudogenes or genomic DNA. Accordingly, the plurality of primers and probes for a set of genes of interest are designed by first determining the location or structure of the introns and exons of each gene of interest. This step of the instant method can be carried out using experimental data and/or any conventional algorithm for determining intron-exon boundaries of a gene (e.g., GENSCAN or GENESPLICER). Such algorithms search the genomic sequence for typical splice sites, e.g., introns end with the dinucleotide ApG (3′ splice site/acceptor) and start with the dinucleotide GpU (5′ splice site/donor). By way of illustration, intron-exon boundaries can be identified by retrieving the genomic sequence of a gene of interest from a database (e.g., the UCSC genome database or NCBI GENBANK database) and comparing the genomic sequence to the mRNA sequence coding for the same protein, which can also be obtained from publicly assessable databases. Such comparisons can be carried out using readily available programs such as BLAT (Kent (2002) Genome Res. 12: 656-664), DIALIGN, CLUSTALW and the like. Stretches of sequences found in the genomic sequences, but not in the mRNA, are indicative of intronic sequences. Exon sequences are then used for primer and probe design, whereas intron sequences are useful for checking for possible interactions during the selection process of primers and probes. The promoter and downstream sequences of each gene can also be retrieved at the users' discretion for sequence comparisons.

Once the exon-intron structure of each gene of interest has been determined, probes specific for spanning exon boundaries (i.e., location where adjacent exons adjoin after the intron has been spliced) are selected. As used herein a “probe” is defined as an oligonucleotide or polynucleotide capable of binding to a target nucleic acid of complementary sequence through one or more types of chemical bonds, usually through complementary base pairing, usually through hydrogen bond formation. As used herein, an oligonucleotide or polynucleotide probe may include natural (i.e., A, G, C, or T) or modified bases (7-deazaguanosine, inosine, etc.). In addition, the bases in oligonucleotide or polynucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages. Oligonucleotide or polynucleotide probes may also be generically referred to as nucleic acid probes.

In particular embodiments, the probes of the present invention are less than 50 bases or nucleotides in length. In certain embodiments the probes are 15 to 50 bases in length, 20 to 40 bases in length, or more specifically 22 to 31 bases in length. As illustrated in FIG. 1B, the probes of the invention are specific for processed RNA, or amplification products thereof, because they anneal to two adjacent exons. In this regard, the probe is a “spanning probe” that anneals to separate (non-contiguous) portions of the gene of interest.

The terms “annealing” and “hybridization” are used interchangeably herein and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding.

In addition to annealing to two adjacent exons, particular embodiments of the present invention embrace the following criteria be used for probe selection:
a. The probe matches its 3′ exon not less than 2+a bp, but not more than 15-b bp, where “a” is the length of the nucleotide sequence shared by the intron and the 3′ exon, and “b” is the length of the nucleotide sequence shared by the intron and the 5′ exon;
b. The probe matches any oligonucleotide in the plurality of primers or probes not more than 9 bp without gap, or 11 bp with a gap at the 3′ end. In other words, the 3′ end of the probe complements less than nine contiguous bases or 11 contiguous bases with a gap of any primer or probe of the plurality of primers and probes;
c. The probe fails to anneal to any amplicon but its own annealing sequence in the set less than 14 bp without gap, or 18 bp with a gap, at its 3′ end. Alternatively stated, the 3′ end of the probe complements at least 14 contiguous bases or 18 contiguous bases with a gap of the gene of interest;
d. The melting temperatures (Tₘ) of the probe is between 50 and 60° C.; and
e. The GC content (GC %) of the probe is between 35% and 70%.

To ensure specificity of the probe for processed RNA, desirably the probe is selected to anneal to exons flanking an intron that is longer than an effectively amplifiable length, e.g., 200, 300 or 400 bases. For each gene of interest, it is desirable that the exons flanking the longest intron are selected for designing primers and probe to achieve the maximal amplification specificity. If no sequences meet the various criteria herein (i.e., GC %, Tₘ, complementarity with target and non-target sequences, etc.), exons flanking the next longest intron can be employed.

To facilitate detection and quantification, particular embodiments of the present invention embrace a labeled probe. The term “labeled probe” refers to a probe that provides a detectable signal. The invention is not limited by the nature of the label chosen. Therefore the label can include, but is not limited to, labels which include a dye or a radionucleotide (e.g., ³²P), fluorescein moiety, a biotin moiety, luminogenic, fluorogenic, phosphorescent, or fluors in combination with moieties that can suppress emission by fluorescence energy transfer (FET). Numerous methods are available for the detection of nucleic acids containing any of the above-listed labels. For example, biotin-labeled probes may be detected using non-isotopic detection methods which employ streptavidin-alkaline phosphatase conjugates. Fluorescein-labeled probes may be detected using a fluorescein-imager. Further the probe may contain positively charged adducts (e.g., the Cy3 and Cy5 dyes) and/or positively charged amino acids to permit detection. In particular embodiments, the probe is selected to be next to a “C,” or other appropriate base, at its 3′ end for the purpose of labeling.

At each junction or boundary of the exons in the RNA, every possible probe sequence is analyzed according to above requirements and criteria, and the selected probe sequence should have the optimal Tₘ (closed to the median of allowed Tₘ range) to achieve the maximal Tₘ uniformity among the plurality of probes.

Once the optimal probe is selected, primers are subsequently selected. The term “primer” refers to a oligonucleotide that anneals to a template nucleic acid sequence and allows synthesis of a sequence complementary to the template nucleic acid. A “pair of primers” refers to primers in a set of at least two primers that are capable of exponentially amplifying a target nucleic acid in the polymerase chain reaction. In particular embodiments, the primers of the present invention are less than 50 bases or nucleotides in length. In certain embodiments the primers are 15 to 50 bases in length, 15 to 40 bases in length, or more specifically 20 to 25 bases in length. As illustrated in FIG. 1B, the primers of the invention are specific for processed RNA, or amplification products thereof, and not unprocessed RNA, pseudogenes or genomic DNA because each primer anneals to a different exon of the gene of interest and is capable of producing an amplifiable product. In accordance with primer selection, the pair of primers can anneal to two adjacent exons or two exons that are close enough to produce an amplifiable product.

The term “amplification product” or “amplicon” as used herein refers to the product of an amplification reaction. Exemplary amplification reactions include, but are not limited to, primer extension, the polymerase chain reaction, and the like. Thus, exemplary amplification products include, but are not limited to, primer extension products, PCR amplicons, and the like. A product is said to be an “amplifiable product” if it is of an appropriate length to be amplified. In accordance with the present invention, an amplifiable product is an amplicon of less than 200 base pairs in length, or more desirably less than 150 base pairs in length. In particular embodiments, the amplicon is 50 to 200 base pairs in length or more desirably, 70 to 150 base pairs in length.

In addition to annealing to two different exons, particular embodiments of the present invention embrace the following criteria be used for primer selection:
a. For each primer at its 3′ end, it matches any other primer in the set by less than 4 bp without gap, or 8 bp with a one-base gap; and in any portion of its sequence it complements any other primer by less than 9 bp without gap, or 12 bp with a gap. Alternatively stated, each primer of the primer pair complements less than nine contiguous bases or 12 contiguous bases with a gap of any other primer of the plurality of primers;
b. The Tₘ of each primer is between 46° C. and 56° C.;
c. The GC content of each primer is between 35% and 70%; and

d. Each primer at its 3′ matches any but its own amplicon in the plurality of primers and probes by less than 14 bp without gap, or 18 bp with a gap. In other words, the 3′ end of each primer complements at least 14 contiguous bases or 18 contiguous bases with a gap of the gene of interest.

When amplifying a plurality of amplicons, primers can be selected such that the lengths of all amplicons in the set differ by at least 7% so that the amplification products can be analyzed by gel electrophoresis when the gene set is small.

While a determination of whether a primer will interact with another primer or probe will minimize the interaction among the selected oligonucleotide sequences and amplicons, other DNA/RNA sequences present in the sample may also be a source of unexpected interaction. Therefore, the present method further includes the step of comparing the sequence of the pair of primers to sequences in one or more databases of genomic or mRNA sequences and determining whether the pair of primers will amplify a non-specific sequence in silico. This step is applied to check for interactions of the selected primers to any genomic and/or RNA sequences in the genome to ensure even higher amplification specificity during multiplex amplification. By way of illustration, primers are aligned to genomic and/or RNA sequences in databases using a program such as BLAST (Altschul, et al. (199) J. Mol. Biol. 215:403-410; Altschul, et al. (1997) Nucleic Acids Res. 25:3389-3402) and it is determined whether non-specific amplification could occur. Non-specific amplification is defined as alignment of the two primers with a non-target genomic or RNA sequence, in the correct orientation for amplification of an amplifiable product. Any primers resulting in non-specific amplification are removed from the set. After a primer is removed because of non-specific amplification, it can be maintained in a database as an “invalid” primer sequence, so that any subsequent primer selection steps will not select the primer again.

In addition to probes and pairs of primers, a nested primer is also selected for generating single-stranded DNA after multiplex amplification. As illustrated in FIG. 1A, the nested primer anneals to the amplicon created from the pair of primers and produces, in an amplification reaction, a single-stranded nucleic acid molecule hybridizable with the probe of the previous steps. The length, GC content, Tₘ and complementarity criteria of the nested primer are the same as those for the probe.

All possible frames of pair or primers and the nested primer flanking the probe are analyzed and the primer sequences with optimal Tₘ are selected. The method of the invention can also be used to design only pairs of primers for a list of genes of interest for multiplex amplification without microarray analysis.

Since the method of the invention selects primers and probes using stringent criteria, a user may list the genes to be included in the analysis in a priority order. In this respect, primers and probes can be designed based on the priority of the genes to ensure the inclusion of genes with higher priorities in the set. It is contemplated that primer and probe selection can start with a new set of genes or add new genes into an existing set. In most cases, multiple rounds of selection may need to be performed for a given set of genes of interest. Each round of selection may only select primers and probes for a fraction of the genes in the set. More round(s) of selection can be performed on top of the previous round to select primers and probes for more genes. The selection cycles can be repeated until a satisfactory number or all of genes are included, or no more genes can be added into the set through the selection.

In so far as the synthesis of oligonucleotide primers and probes is routinely practiced in the art, the primers and probes of the present invention can be synthesized by any conventional or routine method known to those skilled in the art. In addition, it is contemplated that the selection of probes and primers can be carried out by the skilled artisan or can be achieved using a computer executable program, wherein a list of genes of interest is provided as input and a plurality of probes and primers is provided as output.

Subsequent to primer and probe selection and synthesis, gene expression profile analysis is carried out by detecting and/or quantifying the amount of expression products in a sample, i.e., any substance containing nucleic acid material. As illustrated in FIG. 1A, the plurality of primers is used in an amplification reaction in the presence of a sample to produce double-stranded amplicons. The double-stranded amplicons are then contacted with the plurality of nested primers to amplify single-stranded nucleic acid molecules, which are then quantified via hybridization with the plurality of probes. As used herein, the terms “quantitating” or “quantifying” when used in reference to an amplification product, refers to determining the quantity or amount of a particular sequence that is representative of an expression product in the sample. For example, but without limitation, one may measure the intensity of the signal from a labeled probe. The intensity or quantity of the signal is typically related to the amount of amplification product. The amount of amplification product generated correlates with the amount of expression product present prior to amplification, and thus, in certain embodiments, is indicative of the level of expression for a particular gene.

Advantageously, the method of the present invention is highly specific. In contrast to the present invention, conventional high-throughput systems and methods fail to discriminate mRNA from other related DNA and RNA sequences. Using primers amplifying sequences across intron(s) and probes composed of sequences in adjacent exons is a critical enhancement to achieve such high specificity. Furthermore, all primer, probe and amplicon sequences are subjected to exhaustive searches against databases of the entire human genome and transcriptome to ensure these sequences are unique. Experimentally, when genomic DNA was used as samples, signals were only detected for only 2 or 3 genes (0.2%) out of the 1,135 genes. Based on previous studies, these signals may become undetectable in the presence of specific sequences which may compete out the nonspecific amplification.

In addition to being highly specific, the present method is also highly sensitive. It is known that multiplex amplification can detect >1,000 single-copy sequences simultaneously from single haploid sperm cells (Wang, et al. (2005) Genome Res. 15(2):276-283). The fact that >90% of these sequences are detectable indicates that with the specially designed primers herein, most, if not all sequences can be well-amplified in parallel with very limited, if any, interaction among the primers. Since the primers used for gene profiling are designed in the same way, it is reasonable to believe that most gene transcripts are also amplified in parallel. However, since the copy number of different gene transcripts in cells varies widely, the outcome of amplification would be different from that using single-copy sequences. When only single-copy sequences are used in multiplex amplification, most, if not all, sequences may reach the detectable amount before the system is saturated. However, when gene transcripts are amplified, whether a transcript reaches a detectable amount before the system is saturated depends on its copy number in the sample, and not all sequences may reach a detectable amount at the end of amplification. This is likely why some sequences are undetectable by microarray but detectable by gel assay.

Using the method of the invention, a total of 686 gene transcripts were detected from three single cells, which is comparable to 676 for the three 100-cell samples and 693 for all non-single-cell samples from the same cell line. The sensitivity of the method is further proved by the fact that results from 100-cell samples are very similar to each other and to those from 10,000 cells. In addition, specific gene expression profiles are obtained from different cell lines using as few as 100 cells.

The sensitivity of the method is further illustrated by the results showing that a significant portion of transcripts can not be detected from NCI/ADR-RES samples but are detected from the MCF-7 samples or single cell samples, and vice versa. This also indicates that low microarray intensities for these transcripts are not false negatives, and are either not present or present in very low abundance in the respective samples.

In contrast to conventional approaches, the present method is also very simple to carry out. Unlike other methods that involve multiple steps and use multiple enzymes, the method herein can be used to analyze a large number of expression products amplified by a single reverse transcription (RT)-PCR step directly from cell lysates without RNA extraction. In this manner, a large number of samples can be analyzed easily and cost-effectively. The simple experimental procedure is also the basis of the high degree of sensitivity since it avoids complicated RNA extraction and processing procedures before and during amplification, which may cause RNA degradation or loss.

Indeed, when working with RNA, extra precaution has to be taken to prevent RNA degradation. The instant method does not require RNA extraction; once cells are lysed, RNA is directly released to the RT-PCR buffer and used as template immediately. Thus, there is almost no chance for RNase to degrade the RNA templates.

Many studies require the analysis of only a subset of the genes in the human genome and often focus on different gene groups. Therefore, a flexible system is highly desirable. Using the instant method, a large number of expression products can be combined into a single multiplex group. Genes can be easily organized into different subgroups upon need, and can also be re-grouped at any time without altering the reaction conditions. New gene products can be added to an existed set easily.

The capacity of multiplex RT-PCR is another aspect of high-throughput gene expression profiling because it not only makes the amplification of a large number of gene products affordable and cost-effective, but it also eliminates challenges involved in quality control of RT-PCR for a large number of genes individually (Aguilar, et al. (2000) J. Clin. Microbiol. 38(3):1191-1195; Cerveira, et al. (2000) Br. J. Haematol. 109(3):638-640). However, the capacity of multiplex amplification is often limited by interaction between conventional primers. One study reported a screening of 29 expressed genes using multiplex RT-PCR, but was unable to reduce the number of the reaction tubes to less than eight (Pallisgaard, et al. (1998) Blood 92(2):574-588). Other studies achieved multiplexing of up to nine genes with nonspecific RT primers (Malhotra, et al. (1998) Nucleic Acids Res. 26(3):854-856; Tietjen, et al. (2003) Neuron 38(2):161-175). Studies using multiple sets of gene-specific primers in single reactions have also been reported, but none of these generated enough products for the analysis of all expressed genes in the samples (Cerveira, et al. (2000) supra; Clipsham & McCabe (2001) Mol. Genet. Metab. 74(4):435-448). In the present study, success with multiplex RT-PCR for 1,135 mRNA species was achieved. Such a success was based on a combination of several technological developments, including computerized primer design with predicted minimal interaction, a narrow primer Tₘ range, small amplicon sizes, and optimization of amplification conditions. With the current method, it is possible to include two thousand or more gene transcripts in a single multiplex amplification group, and to analyze all human gene transcripts using several multiplexing amplification groups. After pooling amplified products from the multiplexing groups, all genes can be analyzed with a single microarray. With this system, large-scale gene expression profiling becomes highly affordable and cost-effective. If the primers and probes used in the high-throughput analysis are made accessible to the research community through a distribution system, large- and genome-scale gene expression profiling can become even more affordable and cost-effective.

Drug resistance to a broad spectrum of chemotherapeutic agents is a major obstacle in the clinical treatment of human cancer. Therefore, understanding the underlying mechanisms, together with detection of altered gene expression patterns with very sensitive and reliable techniques, is of great importance in increasing the efficacy of cancer therapy. An embodiment of the highly sensitive gene expression profiling system in cancer research is analysis of disseminated tumor cells. Analysis of individual cells is necessary for understanding the early dissemination of tumor cells from a small number of cells or single primary tumor cell. Disseminated tumor cells remaining untouched after complete resection of the primary tumor currently can be detected by bone marrow aspirates. With the highly sensitive method of this invention genetic signature in these cells can be detected, which will provide molecular basis for new therapeutic targets. For example, erbB2 expression has been found to be a therapeutic target for metastatic breast carcinoma. Direct identification of mRNA like that of erbB2 in micrometastatic cells can facilitate the development of an effective therapy, preventing the development of incurable metastasis. In addition, successful analysis of mRNA from microdissected frozen tissue sections without RNA isolation has been demonstrated with human tissue. As such, this invention will be very useful for global gene expression analysis of a biopsy specimen. In addition to cancer research, multiplex RT-PCR with single cells finds application in the study of molecular neurophysiology. This invention can be used to examine the expression of many genes within individual neurons or other cells that can also be characterized with regard to their morphological, electrophysiological and pharmacological features.

The practice of the present invention may employ, unless otherwise indicated, conventional techniques and descriptions of organic chemistry, polymer technology, molecular biology (including recombinant techniques), cell biology, and biochemistry, which are within the skill of the art. Such conventional techniques include polymer array synthesis, hybridization, and detection of hybridization using a label. Specific illustrations of suitable techniques can be had by reference to the example hereinbelow. However, other equivalent conventional procedures can, of course, also be used. Such conventional techniques and descriptions can be found in standard laboratory manuals such as A Laboratory Manual, and Molecular Cloning (from Cold Spring Harbor Laboratory Press); Stryer, Biochemistry, (W H Freeman); and Gait, Oligonucleotide Synthesis: A Practical Approach, 1984 (IRL Press, London).

The present invention also contemplates many uses for oligonucleotides attached to solid substrates. These uses include gene expression monitoring, profiling, library screening, genotyping, and diagnostics. Gene expression monitoring, and profiling are described, e.g., in U.S. Pat. Nos. 5,800,992, 6,013,449, 6,020,135, 6,033,860, 6,040,138, 6,177,248 and 6,309,822. Genotyping and uses therefore are described in U.S. Pat. Nos. 5,856,092, 6,300,063, 5,858,659, 6,284,460 and 6,333,179.

The invention is described in greater detail by the following non-limiting examples.

### EXAMPLE 1

### Materials and Methods

Cell Lines and Single Cell Preparation. Human breast cancer cell line MCF-7 and ovarian cancer cell line NCI/ADR-RES are known in the art (Wu, et al. (2003) Cancer Res. 63(7):1515-1519). The cell lines were maintained in RPMI 1640 medium containing 10% fetal bovine serum, 100 units/ml penicillin, and 100 μg/ml streptomycin at 37° C. in a humidified atmosphere containing 5% CO₂. After counting with a hemacytometer, cells were suspended in PBS (phosphate-buffered saline) to 1000 cells/μl or other desirable densities. Two μl was dispensed into an EPPENDORF tube containing cell lysis buffer (1.5 μl RNase inhibitor, 4 μl of 5× QIAGEN ONESTEP RT-PCR buffer, 12.5 μl H₂O). Single cells were prepared from a diluted cell suspension of 2 cells/μl in 1×PBS. About 0.5 μl of the suspension was pipetted onto a small piece of glass coverslip, and was checked under a microscope. If the droplet contained only one cell, the piece of the coverslip was then transferred into an EPPENDORF tube containing the cell lysis buffer. The tube was immediately frozen in an ethanol/dry ice bath and stored at −80° C. until use.

Selection of Genes for mRNA Profiling. Genes used in the present study were selected for their involvement in fundamental cell functions such as cell cycle, apoptosis, cell matrix, DNA repair, DNA replication, somatic recombination, RNA transcription and regulation, and protein translation and regulation. A complete list of the 1,135 genes of interest and their accession numbers is described by Hu et al. (2008) BMC Genomics 9:9. The borders between exons and introns for the selected genes were determined by aligning of the mRNA to genomic sequences using the BLAT program maintained by the University of California, Santa Cruz.

Primer and Probe Design. A computer program was developed to select primers and probes according the criteria described herein. Each oligonucleotide probe for microarray analysis was designed to be composed of sequences of two adjacent exons to specifically interrogate the cDNA from corresponding mRNA sequence, but not the corresponding genomic sequences or cDNA from unprocessed RNA. To facilitate microarray analysis, the 3′-ends of all probes terminated before a “G” in the template sequence so that they could be labeled with the same fluorescent color by incorporating fluorescently labeled Cy5-ddCTP. The lengths of the probes ranged from 2.2 to 31 bases, and the GC-content of the probes ranged from 30% to 70% with their Tₘ's from 54.4° C. to 65.2° C. A representative list of probes is provided in Table 1.

TABLE 1

| | Accession | | Tₘ | SEQ |
|---|---|---|---|---|
| Gene Symbol | No. | Probe | (° C.) | ID NO: |
| EBF4 | AB037863 | CGGCCGCTTTGTCTACACAGCT | 59.9 | 1 |
| NCOA3 | AF012108 | AGTTTCTCATGGCACTCAAAATAGGCCT | 59.6 | 2 |
| BAG1 | AF022224 | GCCGGGTCATGTTAATTGGGAAAAAGAA | 59.1 | 3 |
| MAP2K7 | AF022805 | TCCAGTCCTTCGTCAAAGACTGCCTTA | 60.2 | 4 |
| PRY2 | AF039843 | CAACGGGTCGCAGCCCTTGCTG | 63.1 | 5 |
| IGFBP5 | AF055033 | TGAGACAGGAGTCTGAGCAGGGC | 60.3 | 6 |
| SCGB2A1 | AF071219 | CTCCTCCTGCACTGCTATGCAGATT | 59.3 | 7 |
| SERF1A | AF073519 | GGCAGTCAAGCTATCCTCTTTCCTCTTT | 59.3 | 8 |
| MS4A7 | AF201951 | CTCTGGAAAACAATCAACTAAGCCCTTTGAC | 59.2 | 9 |
| PECI | AF257175 | ACTCTACGCGCTATATAAGCAGGCCA | 60.1 | 10 |

Each pair of PCR primers was designed to amplify sequences in two adjacent exons flanking a large intron and to ensure specific amplification of the desirable mRNA sequences rather than the respective gene or unprocessed RNA sequences. To enhance the amplification specificity, the program always searched for candidate amplicon sequences separated by large introns in each gene. The melting temperatures (Tₘ's) for all selected primers ranged from 50.1° C. to 61.6° C., and the GC-contents ranged from 32% to 70%. The lengths of the amplicons ranged from 72 to 150 bases. An illustrative list of primer pairs is provided in Table 2.

TABLE 2

| | | | Amplicon | |
|---|---|---|---|---|
| | | Tₘ | Length | SEQ |
| Gene Symbol | Primer | (° C.) | (bp) | ID NO: |
| EBF4 | L, AGCAGTTTTGCAAGGGATGCC | 56.5 | 84 | 11 |
| | R, GTAGCCTCTGGAATCCGTAGTCAA | 56.2 | | 12 |
| NCOA3 | L, GCATGTTGTCCATGGAACAAGTTTC | 55.8 | 118 | 13 |
| | R, GCTCTTTCGTCACTCTGGCCT | 56.8 | | 14 |
| BAG1 | L, TACAAGATGGTTGCCGGGTCA | 56.3 | 93 | 15 |
| | R, CAGACTTCTCCAAATGTTTCAACTT | 52.6 | | 16 |
| MAP2K7 | L, CACATGGGCTTCTCGGGGGA | 58.7 | 120 | 17 |
| | R, CGTCTCGTAGCGCTTGATGAAG | 55.6 | | 18 |
| SPRY2 | L, GCCAGAGCTCAGAGTGGCAA | 56.9 | 102 | 19 |
| | R, CTGGGTGAGGGCGTCTCTGG | 58.8 | | 20 |
| IGFBP5 | L, CGGATCATCTCTGCACCTGAGA | 56.0 | 88 | 21 |
| | R, CTTTGAGCTCCTGCAGGGAAG | 55.3 | | 22 |
| SCGB2A1 | L, CTCATGCTGGCGGCCCTCCT | 61.6 | 80 | 23 |
| | R, ATGGTCTTTTCAACCATGTCCTCC | 55.6 | | 24 |
| SERF1A | L, CTGCTTTCTCTGAGAGGCAGTCA | 56.6 | 87 | 25 |
| | R, GCCCGCCAGAAAAACATGAAGA | 56.4 | | 26 |
| MS4A7 | L, GGATCCCTCTCAATTATCTCTGGAA | 54.1 | 92 | 27 |
| | R, CCTGCAGTAACAGAACTCACTGC | 55.9 | | 28 |
| PECI | L, CAGGAAACGAAGTGAAGCTAAAACT | 54.3 | 90 | 29 |
| | R, AGTCAAATACACCTGGTTTGGGC | 55.9 | | 30 |

L, left primer; R, right primer.

Using the same melting temperature and GC content criteria as for the probes, nested primers were selected. Nested primers were selected for annealing to the amplicons generated by the pairs of primers and for also generating a molecule that would hybridize to probes. An illustrative list of nested pairs is provided in Table 3.

TABLE 3

| | | Tₘ | SEQ |
|---|---|---|---|
| Gene Symbol | Primer | (° C.) | ID NO: |
| EBF4 | TCCGTAGTCAATGGTGGGCTCGTT | 55.6 | 31 |
| NCOA3 | GGCCCAACAAGATCATCCAGGGAA | 55.7 | 32 |
| | TTC | | |
| BAG1 | CAACTTCTTTAGTTCAACCTCTTC | 55.0 | 33 |
| | CTGTGGA | | |
| MAP2K7 | AGCTTATTATACTTTGGTCTCTTC | 52.5 | 34 |
| | CTGTGAT | | |
| SPRY2 | CCCACGCTGTCTGCCACCGTCA | 58.6 | 35 |
| IGFBP5 | GGAAGCCTCCATGTGTCTGCGG | 54.9 | 36 |
| SCGB2A1 | TCTTTTCAACCATGTCCTCCAGGA | 55.2 | 37 |
| | GTTTG | | |
| SERF1A | AGAAAAACATGAAGAAAACCCAGG | 51.6 | 38 |
| | AAATTAG | | |
| MS4A7 | TCACTGCATTTGAGGTCAAGCTGC | 54.9 | 39 |
| | T | | |
| PECI | TGGTTTGGGCATGTTACAAGGTCC | 54.9 | 40 |
| | TT | | |

All primers and probes were subjected to interaction analysis with a computer program developed for designing high-throughput multiplex nucleotide acid detection and all primers were subjected to analysis to determine whether they would amplify an amplifiable product in silico. Probes complementary to intron regions of some genes were also designed as negative controls. All amplicon sequences were subjected to BLAST search to ensure their uniqueness.

Gene-Specific Reverse Transcription and Multiplex RT-PCR. Cells in the lysis buffer described above were lysed with three repeating cycles of alternating one-minute incubations from the ethanol/dry ice mix to a 37° C. water bath before RT-PCR. One-step RT-PCR was carried out in a 50-μl reaction containing primers (20 nM each) for all the 1,135 mRNA species, 2.5 mM MgCl₂, the four dNTPs (400 μM each), and 2.0 μl QIAGEN ONESTEP RT-PCR Enzyme Mix without degenerated primers. The samples were first incubated at 50C for 40 minutes for cDNA synthesis, and then were heated to 95° C. for 15 minutes to inactivate the reverse transcriptase and activate the Taq DNA polymerase followed by 45 PCR cycles. Each PCR cycle included 40 seconds at 94° C. for denaturation, and 1 minute at 55° C. and 5 minutes of ramping from 55° C. to 70° C. for annealing and extension. A final extension step was carried out at 72° C. for 3 minutes at the end of the PCR. All PCRs were performed with the PTC100 Programmable Thermal Controllers (MJ Research). Single-stranded DNA (ssDNA) was generated using the same conditions as in multiplex PCR. Only one primer for each sequence was used, and 40 thermal cycles were carried out.

RT-PCR with Individual Gene Transcripts. RT-PCRs with individual gene transcripts were performed for a group of genes with different amounts of signal intensities detected from the two cell lines, NCI/ADR-RES and MCF-7. For each gene, an aliquot (equivalent of 100 cells) from the same cell lysate used for multiplex gene expression profiling was used. Conditions for one-step RT-PCR were similar to those for multiplex one-step RT-PCR. mRNAs transcribed from β-actin and α-tubulin genes served as internal controls. The PCR products were assayed by gel electrophoresis. Gels were imaged using an Image Station (Model 440, KODAK, New Haven, Conn.). Gel band intensities were digitized with the software, KODAK 1D 3.5.

Microarray Design, Hybridization, and Probe Labeling by Single-Base Extension Assay. Oligonucleotide probes were printed onto glass slides in duplicate with a spot diameter of 160 μm and a center-to-center distance of 250 μm by using the OMNIGRID Accent Microarrayer (Gene Machines, Calif.). One hundred fourteen spots with only microarray printing buffer without probes were used as negative controls and were distributed spatially evenly across each array. Microarray analysis was performed according to established methods (Wang, et al. (2005) supra).

Microarray Scanning and Data Analysis. Microarrays were scanned with a GENEPIX 4000 scanner (Axon Instruments, Foster City, Calif.). The resultant images were digitized with the accompanying software GENEPIX Pro (version 4.0). The mean values of the signals from the duplicate spots of each probe were used for the analysis herein. Background signal was determined by using negative control probes that were complementary to the intron sequences of the corresponding genes or random sequences, and was subtracted from the sample signals. For the comparative expression analysis of the cell lines MCF-7 and NCI/ADR-RES, the array data were normalized by the Lowess smoothing method (Yang, et al. (2002) Nucleic Acids Res. 30(4):e15; Quackenbush (2002) Nat. Genet. 32 Suppl:496-501). After background subtraction, genes with negative intensity values in both duplicated samples were excluded for further analysis. The log ratios of the intensities of the remaining genes in two cells lines were used to make calls and to identify the differentially expressed genes in the samples.

### EXAMPLE 2

### Cancer Gene Expression Array

To establish a cancer gene expression array, a panel of cancer-related genes were selected based on their known functions and/or cancer-associated expression patterns from published literature. All amplicon sequences were subjected to computational screening to ensure their uniqueness. Primers and probes were selected according to a series of criteria described herein. Most primer pairs amplify sequences in two neighboring exons separated by large introns. The intron lengths ranged from 79 bp to 90 kb with an average of 2.0 kb and 97% of the introns are longer than 200 bp. Initially 1,445 genes were used as the input for the primer and probe design program. Primers and probes for 1,120 (77.5%) of these genes were selected. The remaining 22.5% failed in the selection process because of lack of introns or suitable sequences for primers and/or probes. Fifteen of these remaining genes with important functions in cancer development were included in the panel. Primers and probes were designed based on the unique sequences in these genes, and were not required to have introns internally located within the amplified sequences. Therefore, a total of 1,135 genes were included in the multiplex assay.

Microarray-based single-base extension (SBE) assay has been used to genotype single nucleotide polymorphisms (SNPs) (Wang, et al. (2005) supra; Greenawalt, et al. (2006) Genome Res. 16(2):208-214; Hu, et al. (2006) Nucleic Acids Res. 34(17):e116). In the present study, SBE was adapted for gene expression profiling. To simplify the analysis, all probes were designed to terminate immediately before a ‘G’ site in the templates. In this way, the probes were extended by a single base, dideoxynucleoside triphosphate (ddCTP) that was fluorescently labeled. By using one color, the bias associated with different dyes was also eliminated.

To test the reproducibility of the system, gene expression profile analysis was conducted for three duplicated 100-cell samples from an ovarian cancer cell line, NCI/ADR-RES (Liscovitch & Ravid (2007) Cancer Lett. 245(1-2):350-352) and two 100-cell samples from a breast cancer cell line, MCF-7. The data indicated that 660 (58.2%), 663 (58.4%), and 662 (58.3%) gene transcripts were detected from the three 100-cell samples of NCI/ADR-RES, respectively. Of these transcripts, 650 (>98%) were detected from all three duplicates. Signal intensities for the 1,135 genes were strongly correlated between the duplicates (Pearson's r=0.977, 0.974, and 0.949, respectively). Of the 650 transcripts detected in all three NCI/ADR-RES 100-cell samples, only 6 (0.9%), 17 (2.6%), and 1 (0.2%) transcripts had their signal intensities differing by >2 fold between each two of these three duplicates. Twenty-six transcripts were detected from only one or two of the three samples. The signal intensities for these transcripts were low. Only one transcript in one sample had its signal intensity >1,000, indicating that the inconsistence among the duplicates was due to low signals of these transcripts.

For the two 100-cell samples from MCF-7, 615 (54.2%) and 614 (54.1%) gene transcripts were detected, respectively, with 597 (>97%) detected in both. Of these 597 transcripts, 562 (94.1%) had signal intensities differing less than two fold. Similar to the situation with NCI/ADR-RES samples, all 34 transcripts that were detected in only one sample but not the other had low signal intensities with only nine genes whose signal intensities were >1,000 in one of the two samples.

Because samples prepared from a large number of cells are usually associated with high reliability, the microarray results of the NCI/ADR-RES 100-cell samples were further compared with those from a 10,000-cell sample of the same cell line. Resulting data also showed a high degree of correlation (r=0.961). This analysis indicated that 630 (96.7%) of the 650 gene transcripts detected from all the 100-cell samples were also detected from the 10,000-cell sample. Sixty-three gene transcripts were detected in at least one of the three 100-cell samples but not in the 10,000-cell sample, or vice versa. Of these 63 gene transcripts, 61 had signal intensities below 1,000 in all the three 100-cells. However, the change from 100 to 10,000 cells did enhance the detection of 21 gene transcripts whose signal intensities were >2 fold greater in the 10,000-cell sample than those in the 100-cell samples. Among these 21 transcripts, six had signal intensities in the 10,000-cell sample more than 15 fold greater than the average intensities of the corresponding genes in the three 100-cell samples, indicating that using 10,000 cells may have significantly increased the copy numbers of these transcripts or changed their absence status to presence. These data indicated that the system not only could produce very reliable results even with as few as 100 cells but also was very sensitive to the copy number change for the low-copy-number gene transcripts.

To further test the sensitivity of the high-throughput gene expression profiling system, single NCI/ADR-RES cell samples were prepared and used for multiplex gene expression assay of the 1,135 mRNA species. The numbers of gene transcripts detected from the three single-cell samples were 590, 576, and 614, respectively. Of these transcripts, 507 were detected from all single cells. Of the 503, 463 (92.0%) were also detected from all non-single-cell (100-cell and 10,000-cell) samples, indicating a prevalent expression of these genes in most, if not all, cells at relatively high levels.

A wider range of gene transcripts was detected from the three single-cell samples compared to the non-single-cell samples. Four hundred forty-nine transcripts were undetectable in all three single cell samples, a number which is not greater than that (459) for the three 100-cell samples and is comparable to that (442) for all non-single-cell samples. The number of undetectable gene transcripts from all single and non-single cell samples is 357. This number means that from single cells, a comparable number of genes were not only detected, but also a new set of 92 genes were detected that could not be detected with non-single-cell samples of the same cell line.

The robustness of gene expression profiling with single-cell samples was also demonstrated by the signal intensities. As described above, most transcripts that were detected from some but not all non-single-cell samples had low signal intensities with very few >1,000. The scenario with single cells was very different. Of the 503 gene transcripts detected from all single cells, 40 were detected in one to three non-single-cell samples but not all four. All 40 but one had signal intensity >1,000 in at least one of the three single-cell samples. Of the 183 transcripts that were only detected from one or two single-cell samples, 108 (59.0%) had signal intensity >1,000. The strong and robust signal intensities detected from single-cell samples indicated that the method was very sensitive.

Unlike the gene transcripts detected from all non-single-cell samples which account for more than 95% of gene transcripts detected from each of these samples, the 503 gene transcripts detected from single cells only account for 85. 3%, 87.3% and 81.9% of the transcripts detected from individual single-cell samples, respectively. Pairwise comparison of the results from the single-cell samples yielded correlation coefficients of 0.780, 0.700, and 0.711, respectively compared with 0.949 or greater for other non-single-cell samples. From all single and non-single-cell samples, 778 gene transcripts were detected, of which 315 (40.5%) were detected from some but not all samples. This is in contrast with the scenario of non-single-cell samples from which gene transcripts that were only detected from some but not all samples were a very small portion. Furthermore, of these 315 transcripts, 177 (56.2%) were either detected from only single cells or from non-single cell samples.

The high degree of concordance among the results from the non-single-cell samples, and the significant differences among those from single cells, and between single cells and non-single-cell samples indicate that most, if not all, of these differences are real. As mentioned above, this is further supported by the robustness of the signal intensities detected from single-cell samples for the gene transcripts that were detected differently between the single cells and non-single-cell samples. It is conceivable that heterogeneity in clonality and/or genetic alterations in the cells of a cell line could be major factors contributing to the differences. In addition, a considerable portion of the cells may be at different cycle stages during which groups of genes are expressed differently. Therefore, while gene expression in single cells could differ in various aspects, 100 cells may well represent the entire cell population because, after all, the cell line cells are from the same tissue and the same donor. Therefore, genes that are detectable in a cell population may not be expressed or expressed at very low levels in certain single cells. Conversely, genes that are detectable in particular single cell samples may not be expressed or expressed at very low levels in the majority of the cell population.

When the gene expression profiles of NCI/ADR-RES were compared with those of MCF-7, a considerable number of genes were shown to be expressed differentially in these two cell lines. Of the 1,135 gene products, 531 (46.8%) were detected from samples of both cell lines (not including single cell samples). Seventy-five gene transcripts were detected in all NCI/ADR-RES non-single-cell samples, but not in the MCF-7 samples, and 43 were detected in the opposite way.

The specificity of the high-throughput gene expression method was demonstrated by the results from different cell line samples and by those from different single cells as described above. To further demonstrate the specificity of the system, human genomic DNA samples were amplified with the same multiplex RT-PCR procedure and analyzed by microarray. Very few probes (<0.2%) were shown to have signals above the background, indicating that the method was very specific and could discriminate between the target mRNA sequences from their genomic counterparts, and therefore, the unprocessed transcripts.

What is claimed is:

1. A method for high-throughput gene expression profile analysis comprising
(I) synthesizing a plurality of primers and probes for a set of genes of interest by:
(a) determining the intron-exon structure of each gene of interest;
(b) selecting for each gene of interest a probe, wherein said probe:
(i) is less than 50 bases in length, and
(ii) anneals to two adjacent exons flanking an intron of at least 200 bases in length;
(c) subsequently selecting for each gene of interest a pair of primers, wherein said primers:
(i) are less than 50 bases in length,
(ii) anneal to two exons of the gene of interest,
(v) produce an amplicon of less than 200 base pairs in length, and
(vi) when compared to sequences in one or more databases of genomic or mRNA sequences, fail to amplify a non-specific sequence in silico; and
(d) subsequently selecting a nested primer, wherein said nested primer:
(i) anneals to the amplicon of step (c)(ii), and
(ii) produces, in an amplification reaction, a single-stranded nucleic acid molecule hybridizable with the probe of step (b); and
(II) subjecting a sample to an amplification reaction with the plurality of primers of step (I)(c), thereby producing double-stranded amplicons;
(III) amplifying single-stranded nucleic acid molecules from the double-stranded amplicons with the plurality of nested primers of step (I)(d); and
(IV) quantifying the single-stranded nucleic molecules via hybridization with the plurality of probes of step (I)(b) thereby determining the expression profile of the set of genes of interest in the sample.

2. The method of claim 1, wherein said probe has a melting temperature of between 50° C. and 60° C., has a GC content of between 35% and 70%, complements less than nine contiguous bases or 11 contiguous bases with a gap of any primer or probe of the plurality of primers and probes, and complements at least 14 contiguous bases or 18 contiguous bases with a gap of the gene of interest.

3. The method of claim 1, wherein the primers of said pair of primers have a melting temperature of between 46° C. and 56° C., have a GC content of between 35% and 70%, complement less than nine contiguous bases or 12 contiguous bases with a gap of any other primer of the plurality of primers, and complement at least 14 contiguous bases or 18 contiguous bases with a gap of the gene of interest.

4. The method of claim 1, wherein the probe is labeled.
